(19) United States
(12) Patent Application Publication
(10) Pub. No.: US 2012/0232250 A1
(43) Pub. Date: Sep. 13, 2012

(54) METHOD FOR PRODUCING A RADIOACTIVELY MARKED PEPTIDE

(76) Inventors: Markus Kinzl, Dietzenbach (DE); Oliver Lade, Berlin (DE); Arno Steckenborn, Berlin (DE)

(21) Appl. No.: 13/387,918

(22) PCT Filed: Jul. 7, 2010
(86) PCT No.: PCT/EP10/59730

§ 371 Date: May 14, 2012

(30) Foreign Application Priority Data
Jul. 29, 2009 (DE) 10 2009 035 645.2

(51) Int. Cl. C07K 1/13; C07K 2/00

(52) U.S. Cl. 530/345

(57) ABSTRACT

A method for producing a radioactively marked peptide, uses a precursor molecule that is prepared in an organic solvent. A radioactively marked compound having a carboxyl function is added. The carboxyl function is activated, and the activated radioactively marked compound is bonded to the precursor molecule in order to form the radioactively marked peptide. The radioactively marked compound is an isocyanocarboxylic acid. A radioactively marked isocyanocarboxylic acid is used for producing a radioactively marked peptide.

### CROSS REFERENCE TO RELATED APPLICATIONS

This application is the U.S. national stage of International Application No. PCT/EP2010/059730, filed Jul. 7, 2010 and claims the benefit thereof. The International Application claims the benefits of German Application No. 10 2009 035 645.2 filed on Jul. 29, 2009, both applications are incorporated by reference herein in their entirety.

### BACKGROUND

In the technical field of radioactively labeled carbon compounds, described below is a method for producing a radioactively labeled peptide and also a use of a radioactively labeled isocyanocarboxylic acid for producing a radioactively labeled peptide.

The development of solid-phase and liquid-phase chemistry now makes possible the production of sequence-defined peptides and proteins having molecular weights of 3000-10 000 Da and coupling yields of greater than 99.5%. In the solid-phase synthesis devised in 1963 by Merrifield, the peptide that is to be synthesized is coupled via a linker i.e. a cleavable anchor group, to an insoluble carrier resin made of crosslinked polymer. The amino acids are successively coupled in the desired sequence to an activated C-terminus and in great excess to the amino function of the last attached amino acid in each case. Remaining reactants and byproducts can be flushed from the reaction vessel, since the intermediate or product is bound to the insoluble resin. In the last step, the linker is cleaved from the resin, and so the peptide is present in free form.

Both solid-phase and liquid-phase peptide synthesis are based on a complex protecting group chemistry. The alpha-amino protecting group of the amino acid must be temporarily protected for the coupling, since the amino acid, after activation of the carboxylic acid, would otherwise react with itself. After the coupling, this protecting group must be eliminated rapidly and under mild conditions, in order that further coupling can proceed.

Peptides and proteins that are physiologically active in the organism and in the chemical structure of which one or more radionuclides are incorporated, provide the basis for producing radiopharmaceuticals. The organism does not differentiate, at least in the case of chemically identical radiopharmaceuticals, between radiopharmaceuticals and the corresponding nonradioactively labeled compounds, and so radiopharmaceuticals are metabolized physiologically. On the basis of the decay of the radionuclide, the radiopharmaceutical can be traced and displayed visually.

Radioactively labeled peptides are valuable tracers for positron-emission tomography (PET), a method in nuclear medicine which generates sectional images of living organisms. In PET, from the distribution in time and space of the recorded decay events, conclusions can be made as to the spatial distribution of the radiopharmaceutical in the body and processes such as absorption, distribution, metabolism and excretion can be illustrated.

The applicability of radiopharmaceuticals is limited by the short half life of the radionuclides of typically less than 2 hours. A particularly short half life is possessed by the radionuclide ¹¹C, having only approximately 20 minutes. The unwanted decrease in radioactivity already begins on production of the radionuclide in the cyclotron and continues during production of the radiopharmaceutical, its delivery to the PET site, and finally up to administration to the patient and measurement.

In order to achieve the largest possible delivery radius, in which the positron emission tomograph to be delivered is situated, around the cyclotron, a radioactivity as high as possible of the radiopharmaceutical must be present after production thereof. This can be achieved by a very short production time of the radiopharmaceutical from the radionuclide, since the loss in radioactivity for a given radionuclide depends on time.

However, most radio-labeling methods for peptides are multistage, time-consuming, difficult to automate and only succeed with low radiochemical yields. Conventional methods for producing radiopharmaceuticals use the pathway via the methylation agent ¹¹CH₃I in order to label, for example, amines or carboxylic acids and amino acids radioactively with ¹¹C (Denutte et al;, radioactive 1983; Vandersteene and Sledgers, 1996). However, in this case, the “CO₂ that is produced in the cyclotron must be further reacted in a two-stage process with LiALH₄ and HI to form ¹¹CH₃I. Not until a third stage can the radioactively labeled methylation agent be transferred to the pharmaceutical that is to be labeled. By way of this lengthy synthesis of the radiopharmaceutical, a high proportion of the radioactivity originally provided by ¹¹CO₂, is lost.

An important technology for novel PET contrast agents was introduced in the field of ¹⁸F-labeling by what is termed “click chemistry”. This method permits the synthesis of radioactive contrast agents in a single step (Devaraj et al., 2009; Li et al., 2007). In the work by Bruus-Jensen (2006), HYNIC-functionalized peptides and proteins are used as precursors for synthesizing ¹⁸F- and ⁹⁹mTc-labeled radiopharmaceuticals. Radiopharmaceuticals labeled with technetium or fluorine such as, e.g. ¹⁸F-6-fluoro-DOPA or ¹⁸F-fluoro-2-deoxy-D-glucose, differ, however, in the organism from the analogous unlabeled original molecules thereof.

### SUMMARY

One possible object is to provide an efficient and rapid synthesis method which, within a short production time, delivers a high yield of radioactively labeled natural and artificial peptides.

The inventors propose a method for producing a radioactively labeled peptide, comprising the following steps:
(a) providing a precursor molecule which is selected from the group consisting of an amino acid, a peptide and a primary amine, in an organic solvent;
(b) adding a radioactively labeled compound to the precursor molecule which comprises a carboxyl function;
(c) activating the carboxyl function of the radioactively labeled compound; and
(d) linking the activated radioactively labeled compound to the precursor molecule to give the radioactively labeled peptide, wherein the radioactively labeled compound is an isocyanocarboxylic acid.

In addition, the inventors propose a use of a radioactively labeled isocyanocarboxylic acid for producing a radioactively labeled peptide. BRIEF DESCRIPTION OF THE DRAWINGS

These and other aspects and advantages will become more apparent and more readily appreciated from the following description of the exemplary embodiments, taken in conjunction with the accompanying drawings of which:

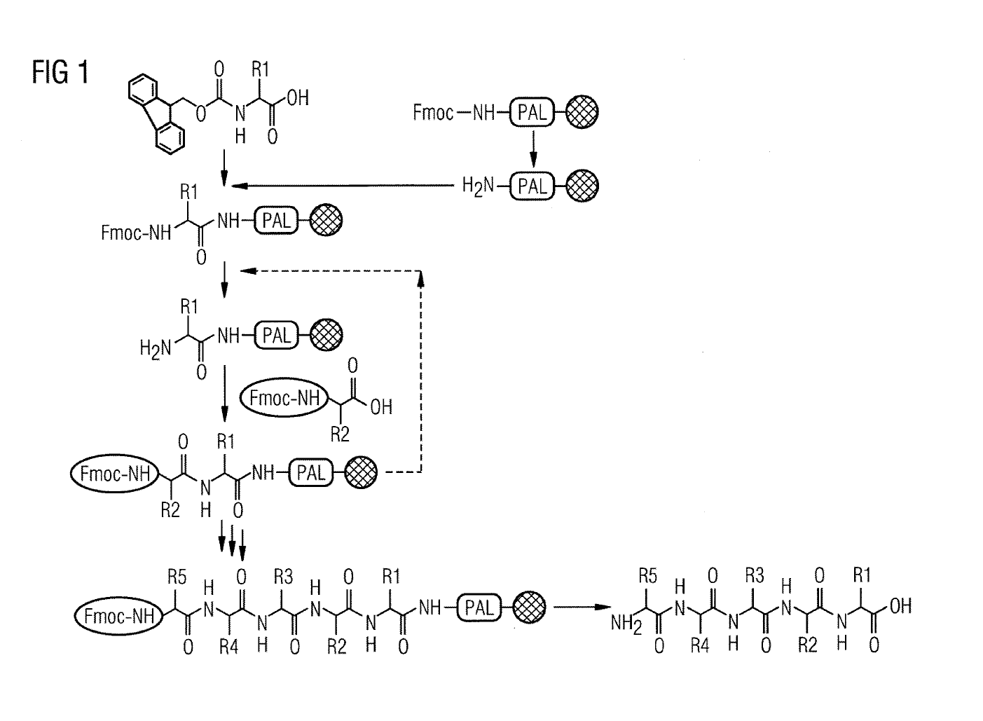

FIG. 1 shows a conventional solid-phase peptide synthesis using amino acids, the amino function of which is blocked by the protecting group 9-fluorenylmethoxycarbonyl (Fmoc).

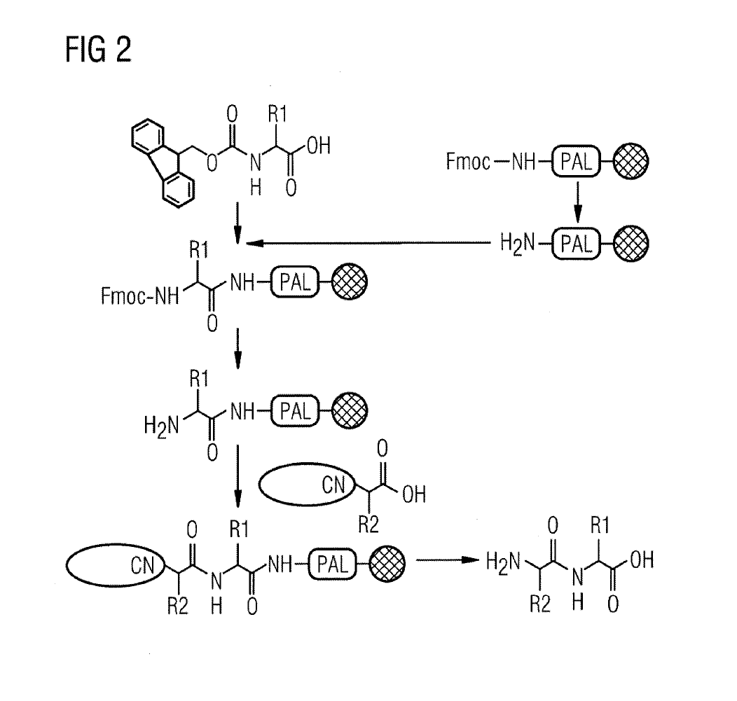

FIG. 2 shows a solid-phase peptide synthesis according to the inventors' proposals, using a labeled isocyanocarboxylic acid.

### DETAILED DESCRIPTION OF THE PREFERRED EMBODIMENT

Reference will now be made in detail to the preferred embodiments, examples of which are illustrated in the accompanying drawings, wherein like reference numerals refer to like elements throughout.

The proposed method for producing a radioactively labeled peptide, may include:
(a) providing a precursor molecule which is selected from the group consisting of an amino acid, a peptide and a primary amine, in an organic solvent;
(b) adding a radioactively labeled compound to the precursor molecule which comprises a carboxyl function;
(c) activating the carboxyl function of the radioactively labeled compound; and
(d) linking the activated radioactively labeled compound to the precursor molecule to give the radioactively labeled peptide, wherein the radioactively labeled compound is an isocyanocarboxylic acid.

The expression “peptide”, as used here, denotes an organic compound which is made up of at least two amino acids linked to one another via an amide or peptide bond. The expression “peptide” comprises oligopeptides of up to 10 amino acids, polypeptides of more than 10 amino acids and macropeptides of more than 100 amino acids, and also proteins independently of the primary, secondary, tertiary and quaternary structure. Peptides comprise artificial and naturally occurring organic compounds. They can be produced chemically and also by biosynthesis.

The expression “precursor molecule”, as used here, denotes monomers, oligomers and polymeric starting molecules of the peptide synthesis which comprise amino acids, peptides and/or primary amines of the formula RNH₂.

The expression “carboxyl function”, as used here, denotes a functional group of the carboxylic acid having the formula —COOH, or of the carbonate having the formula —COO⁻.

The expression “activating the carboxyl function”, as used here, denotes converting a carboxylic acid into a reactive substance.

The expression “isocyanocarboxylic acid”, as used here, denotes an organic compound which comprises a carboxyl group, —COOH, or a carboxylate, —COO⁻, and an isocyano group, —CN. The isocyanocarboxylic acid possesses, e.g., the empirical formula CNR₁R₂CCOOH or CNR₁R₂CCOOX.

The radicals R, R₁, R₂ etc., as used here, denote identical or different aromatic, heteroaromatic and aliphatic radicals and also nitrogen compounds, halogen compounds and hydrogen groups. The aliphatic radicals comprise acyclic branched and unbranched, cyclic and alicyclic, saturated and unsaturated carbon compounds. X comprises metal ions, such as alkali metal and alkaline earth metal ions, e.g. lithium.

For producing a radioactively labeled peptide, in the method according to the invention, radioactively labeled isocyanocarboxylic acid is used. The isocyano group of the isocyanocarboxylic acid must not be blocked by a protecting group in the peptide synthesis—in contrast to the amino group of an amino acid. Therefore, valuable reaction time is saved which is required during the conventional synthesis by way of linking of amino acids in order to 1.) add the protecting group in an additional reaction step to the amino group and 2.) to eliminate the protecting group again in a further reaction step after linking the amino acid to the peptide. Therefore, the time taken for peptide synthesis is shortened by way of the method.

By way of the shortened peptide synthesis time of the proposed method, radiochemical yield losses due to the natural decay of the radionuclide as a function of time are decreased. The amount of radioactive starting substance used in the synthesis of the radioactively labeled peptide can therefore be decreased, which saves costs, and the burden of the radiochemist during the synthesis is reduced.

The production of radioactively labeled peptide by way of radioactively labeled isocyanocarboxylic acid is very simple and may be carried out with relatively low expenditure. The method can therefore be employed directly in the clinic or radiological practice.

In a preferred embodiment, the organic solvent comprises methylene chloride, chloroform, dichloroethane, dimethylformamide, dimethylacetamide, tetrahydrofuran, ethyl acetate, acetonitrile and/or a combination thereof.

In a further preferred embodiment, the radioactively labeled isocyanocarboxylic acid comprises radioactively labeled carbon, preferably ¹¹C.

The isocyanocarboxylic acid is produced by carboxylating CNR₁R₂CH or CNR₁R₂CX using radioactively labeled carbon dioxide, e.g. ¹¹CO₂. The incorporation of the radionuclide therefore proceeds in the last step of synthesizing the radioactively labeled isocyanocarboxylic acid, which can be used directly in the peptide synthesis according to the proposals. Therefore, already in the synthesis of isocyanocarboxylic acid, reaction time is saved, whereby radiochemical yield losses due to decay of the radionuclide are decreased and the amount of radioactively labeled isocyanocarboxylic acid used is decreased. In turn, costs are saved thereby and the burden on the radiochemist during the peptide synthesis is decreased.

In a preferred embodiment, the method additionally comprises :
(a1) adding a further amino acid or an unlabeled isocyanocarboxylic acid, each of which comprises a carboxyl function, to the provided precursor molecule;
(a2) activating the carboxyl function of the further amino acid or the unlabeled isocyanocarboxylic acid;
(a3) linking the provided precursor molecule to the further amino acid or the unlabeled isocyanocarboxylic acid via an amide bond to form a peptide; and
(a4) repeating steps (a1) to (a3) until a desired size of peptide is achieved.

In steps (a1) to (a4) of the embodiment, subsequently to step (a) of the method, oligomeric and polymeric precursor molecules are synthesized, such as oligopeptides and polypeptides, by adding further monomeric amino acids to the precursor molecule, i.e. the amino acid, the peptide or the primary amine. The radioactively labeled isocyanocarboxylic acid can therefore be added to precursor molecules of any desired length in the method (cf. step (b) above).

In a preferred embodiment, the isocyanocarboxylic acid is an alpha-isocyanocarboxylic acid. The expression “alpha-isocyanocarboxylic acid”, as used here, denotes an organic compound which comprises a carboxyl group or a carboxylate, and an isocyano group on the same carbon atom (IUPAC name: 2-isocyanocarboxylic acid).

In a preferred embodiment, activating the carboxyl function comprises converting the isocyanocarboxylic acid and/or the further amino acid into a reactive substance which comprises active ester, anhydride, pentafluorophenyl ester, thioester, imidazolide, acyl halide and/or dimethylaminopyridine.

In a further preferred embodiment, the isocyanocarboxylic acid is reacted with a coupling reagent to give the active ester, wherein the coupling reagent comprises guanidinium reagent, a uronium reagent, preferably 2-(H-benzotriazol-1-yl)-1,1,3,3-tetramethyluronium hexafluorophosphate (HBTU), O-(benzotriazol-1-yl)-N,N,N′,N′-tetramethyluronium tetrafluoroborate (TBTU) or 2-(1H-7-azabenzotriazol-1-yl)-1,1,3,3-tetramethyluronium hexafluorophosphate (HATU), a benzotriazole reagent, preferably 1-hydroxybenzotriazole reagent (HOBt), an immonium reagent, a carbodiimide reagent, preferably N,N′-dicyclohexyl carbodiimide (DCC) or diisopropyl carbodiimide (DIPCDI), an imidazolium reagent, an organophosphorous reagent, an acidic halogenating reagent, a phosphonium reagent, preferably benzotriazol-1-yl-oxy-tris(dimethylamino)phosphonium hexafluorophosphate (BOP); also known as Castros reagent) or benzotriazol-1-yl-oxytripyrrolidinophosphonium hexafluoro-phosphate (PyBOP), a morpholine reagent, preferably N-methylmorpholine (NMM), a chloroformate reagent and/or a combination thereof.

HOBt is advantageous, because it accelerates the formation of the peptide bond, suppresses the racemization and has the effect that, e.g., the Asn and Gln side groups do not dehydrate. Using DCC, the amino acids may be converted in situ into active esters which are sufficiently stable that they can be isolated and chromatographed.

Particularly preferably, a mixture of HOBt and DCC is used. DCC activates the carboxylic acid with formation of a highly reactive acyl isourea. This reacts with the HOBt to form an HOBt-active ester which preserves a very large amount of the initial reactivity. The HOBt-active ester is nucleophilically attacked by the amino function of the peptide or a further amino acid. With the elimination of water, a peptide bond is formed. A direct reaction of the acyl isourea is not very unadvisable, since the reactivity is sufficiently high that racemization occurs. The addition of DIPEA to HOBt acts as a catalyst which reduces the tendency to racemization of HOBt.

Alternatively to the DCC, DIPCDI is frequently used together with HOBt, since the urea that forms in this reaction is more soluble and can be more readily separated off.

Particular preference is given in addition to BOP or a mixture of HBTU and HOBt. It is important that the counterion of BOP or HBTU is of very low nucleophilicity, such as, e.g., PF⁻. The BOP reagent is stable, non-hygroscopic and is very readily soluble in organic solvents. In general, the BOP reagent is more efficient than the combination DCC/HOBt. A disadvantage of the BOP reagent, however, is that during the reaction, carcinogenic hexamethylphosphoric triamide is formed. The novel reagent PyBop, in contrast, does not exhibit carcinogenicity. This reagent possesses pyrrolidine units instead of methyl groups.

The reagents HBTU and HATU have a particularly high reactivity. Acyl chlorides and acyl fluorides are readily available and inexpensive reagents.

In a preferred embodiment, a function of a side chain of the precursor molecule, of the isocyanocarboxylic acid and/or of the further amino acid is blocked by a protecting group. The function comprises a hydroxyl function, a carboxyl function and an amino function.

In a preferred embodiment, a carboxyl function and/or amino function of a main chain of the precursor molecule and/or an amino function of a main chain of the further amino acid is/are blocked by a protecting group.

The expression “protecting group”, as used here, comprises compounds which block and switch off the reactivity of chemical groups in a targeted manner by binding to these chemical groups. The expression “side chain”, as used here, comprises the radicals R₁, R₂ etc., which branch off from the main strand of the precursor molecule, the isocyanocarboxylic acid or the further amino acid. The expression “main chain”, as used here, denotes the N- and C-terminal backbone which forms the axis or stem of the precursor molecule, the isocyanocarboxylic acid or the further amino acid.

In a preferred embodiment, the protecting group comprises a base-stable protecting group, a t-butyloxycarbonyl protecting group (Boc) and/or a 9-fluorenylmethoxycarbonyl protecting group (Fmoc).

If the N terminus is protected by Fmoc, then, e.g. base-stable, acid-labile, protecting groups are used for the side chains. Examples thereof are Boc, which protects the amino function, e.g. in lysine; tert-butyl, which protects the carboxyl groups and hydroxyl groups, e.g. in aspartic acid and serine; and trityl, which protects amides, e.g. in glutamine.

In a preferred embodiment, the method additionally comprises :
(a1) eliminating the protecting group from the amino function of the main chain of the provided precursor molecule;
(a2) adding the further amino acid, the amino group of the main chain of which is protected, to the precursor molecule;
(a3) activating the carboxyl function of the main chain of the further amino acid
(a4) linking the precursor molecule to the further amino acid via an amide bond to the peptide; and
(a5) repeating steps (a1) to (a4) until a desired size of the peptide is achieved.

In steps (a1) to (a5) of the embodiment, subsequently to step (a) of the method, oligomeric and polymeric precursor molecules are synthesized, such as oligopeptides and polypeptides, by adding further monomer amino acids to the precursor molecule, i.e. the amino acid, the peptide or the primary amine. The N-terminal amino function of the precursor molecule is blocked by the eliminatable protecting group. The radioactively labeled isocyanocarboxylic acid can therefore be added, in the method, to precursor molecules of any desired length that are equipped with protecting groups (cf. step (b) above).

In a preferred embodiment, the Fmoc protecting group is eliminated by ammonia, a primary amine or a secondary amine, preferably 4-aminomethylpiperidine, piperidine or tris(2-aminoethyl)amine.

In a preferred embodiment, the t-butyloxycarbonyl protecting group is eliminated by protons.

In a preferred embodiment, the method additionally comprises the step of hydrolyzing the isocyanocarboxylic acid linked to the precursor molecule. The isocyano group is thereby converted into a functional amino group. The peptide synthesis can be terminated or continued at this amino group. The radionuclide can therefore be situated within the peptide chain or at one end of the peptide chain. In addition, the radioactively labeled peptide synthesized can comprise one or more radionuclides.

In a further preferred embodiment, the method additionally comprises the step of eliminating the protecting group from the radioactively labeled peptide. After completion of the peptide synthesis, e.g. conditionally acid-stable protecting groups are eliminated by hydrogen halides such as HF, and acid-labile protecting groups are eliminated by trifluoroacetic acid (TFA).

In a preferred embodiment, the precursor molecule is coupled to a solid phase.

The expression “solid phase”, as used here, denotes a polymeric solid support to which the peptide is bound during synthesis thereof. By way of this immobilization, the substances used can be added in great excess and washed out very rapidly, whereby the coupling yield in the method is markedly increased. The solid phase chemistry additionally makes it possible to automate the peptide synthesis. For instance, in the peptide synthesis, on the solid phase, sequential repetition of the steps of adding the precursor molecules, monomers and reagents, activating the carboxyl function, linking of precursor molecule and monomer and eliminating the temporary protecting group is carried out.

The solid phase comprises a linker which is the binding element between the polymeric support and the peptide.

In a particularly preferred embodiment, the solid phase is a polystyrene resin, a 2′,4′-dimethoxyphenylhydroxymethylphenoxy resin, a p-methylbenzhydrylamine resin, a phenalacetamidomethyl resin and/or an oxime resin.

Polystyrene resin swells readily, and so the reagents can readily arrive at the synthesis site. In addition, it is inert towards the reagents. The polystyrene is preferably admixed with 1% m-divinylbenzene for crosslinking. The functionalization is achieved, e.g., via a chloromethylation. Between the chloromethyl group and the first amino acid, advantageously a linker is placed, e.g. a p-alkoxybenzyl ester linker, which permits the elimination of the finished peptide from the resin at the end of the synthesis. Polystyrene resins having alkoxybenzyl ester linkers are preferably used in the context of the Fmoc-peptide synthesis, make possible the synthesis of C-terminal carboxylic acids and are produced by reacting the chloromethyl polystyrenes with 4-hydroxybenzyl alcohol. The finished peptide is cleaved using TFA.

2′,4′-Dimethoxyphenylhydroxymethylphenoxy resins are used either as amide or acid resins. The amide resin delivers C-terminal amides and is used for the Fmoc-synthesis.

The acid resin makes possible a peptide synthesis by way of the N-terminal Boc protecting group. It delivers C-terminal carboxylic acids. The linker of the acid resin is, just as is the chlorotrityl linker, sufficiently acid-labile that the peptides can be cleaved in protected form from the resin, e.g. using dilute TFA. These fragments can then be used in a fragment condensation.

p-Methylbenzhydrylamine resins and phenalacetamidomethyl resins are used in the context of Boc-peptide synthesis. The elimination of the finished peptides proceeds at the end using HF. Using the p-methylbenzhydrylamine resins, peptide amides are obtained. The phenalacetamidomethyl resins deliver carboxylic acids.

By way of oxime resins, completely Boc-protected peptides can be produced. The elimination takes place using NH₃ or H₂N—NH₂.

In a preferred embodiment, the method additionally comprises the step of decoupling the radioactively labeled peptide from the solid phase.

In order to liberate the radioactively labeled peptide, after completion of the peptide synthesis, it is decoupled from the solid phase or the linker of the solid phase. In the case of the N-terminal Boc-protecting group, the elimination is achieved using HF, and in the case of the N-terminal Fmoc-protecting group, using approximately 80% strength TFA. In addition to these acids of different strength, e.g. nitrobenzyl resins are eliminated with light and allyl resins with Pd(O) orthogonally.

In a preferred embodiment, the hydrolysis takes place simultaneously with the decoupling of the precursor molecule from the solid phase.

In a further preferred embodiment, the hydrolysis takes place simultaneously with the decoupling of the radioactively labeled peptide from the solid phase and the elimination of the protecting group from the radioactively labeled peptide.

The simultaneous decoupling and hydrolysis, or decoupling, hydrolysis and elimination, decreases the process time and therefore reduces radiochemical yield losses. Therefore, the amount of radioactive isocyanocarboxylic acid used in the synthesis can be decreased.

The simultaneous hydrolysis and decoupling of the radioactively labeled peptide from the solid phase and also the elimination of the Boc protecting group is achieved using liquid HF, trifluoromethanesulfonic acid in trifluoroacetic acid or HBr in acetic acid. In the case of the Fmoc protecting group, TFA (approximately 80% strength) in CH₂Cl₂ is used. Preferably, trapping reagents such as anisole, ethanedithiol or dimethylsulfide are added, in order to trap reactive intermediates which can damage the peptide.

In a further aspect, the invention relates to the use of a radioactively labeled isocyanocarboxylic acid for producing a radioactively labeled peptide.

FIG. 1 shows a conventional solid-phase synthesis for producing peptides. The precursor molecule is a primary amine protected by Fmoc which is coupled to a solid phase (double hatched circle) via an aminomethyl-3,5-dimethoxyphenoxyvaleryl linker (PAL). Fmoc is eliminated from the amine by a base, and an added amino acid is activated as HOBt ester. Then, the amino acid, the amino group of which is likewise protected by Fmoc, is linked to the immobilized amine. In a further step, the Fmoc group is in turn eliminated from the amino group of the immobilized precursor molecule, and a further added and activated amino acid is linked to the precursor molecule. These steps are repeated until the desired length of the peptide is achieved. Finally, the protecting groups are eliminated and the peptide is decoupled from the solid phase.

FIG. 2 shows schematically a synthesis according to the proposals, of a radioactively labeled peptide by way of radioactively labeled isocyanocarboxylic acid. First, a precursor molecule is synthesized conventionally from a Fmoc amino acid and an amine coupled to a solid phase (cf. FIG. 1). An activated radioactively labeled alpha-isocyanocarboxylic acid is attached to the precursor molecule, the CN group of which alpha-isocyanocarboxylic acid need not be protected, which saves reaction time. The CN group is then hydrolyzed to give the NH₂ amino group and simultaneously the synthesized radioactively labeled peptide is decoupled from the solid phase.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

1-21. (canceled)

22. A method for producing a radioactively labeled peptide, comprising:
providing a precursor molecule which is selected from the group consisting of an amino acid, a peptide and a primary amine, in an organic solvent;
adding a radioactively labeled compound to the precursor molecule in the organic solvent, the radioactively labeled compound being an isocyanocarboxylic acid, the radioactively labeled compound having a carboxyl function;
activating the carboxyl function of the radioactively labeled compound to produce an activated radioactively labeled compound; and
linking the activated radioactively labeled compound to the precursor molecule to produce the radioactively labeled peptide.

23. The method as claimed in claim 22, wherein the organic solvent is selected from the group consisting of methylene chloride, chloroform, dichloroethane, dimethylformamide, dimethylacetamide, tetrahydrofuran, ethyl acetate, acetonitrile and a combination thereof.

24. The method as claimed in claim 22, wherein the radioactively labeled compound is isocyanocarboxylic acid radioactively labeled with ¹¹C.

25. The method as claimed in claim 22, wherein the following steps are repeated until a desired size of peptide is achieved:
hydrolyzing the activated radioactively labeled compound linked to the precursor molecule, to form an amino group from an isocyano group of the isocyanocarboxylic acid and produce a new precursor molecule;
adding a further amino acid comprising a carboxyl function, or an unlabeled isocyanocarboxylic acid comprising a carboxyl function, to the new precursor molecule;
activating the carboxyl function of the further amino acid or the unlabeled isocyanocarboxylic acid; and
after activating the carboxyl function, linking the new precursor molecule to the further amino acid or the unlabeled isocyanocarboxylic acid via an amide bond to form a peptide

26. The method as claimed in claim 25, wherein
the unlabeled isocyanocarboxylic acid is added to the new precursor molecule,
linking the new precursor molecule to the unlabeled isocyanocarboxylic acid produces a new linked compound, and
after linking the new precursor molecule to the unlabeled isocyanocarboxylic acid, the new linked compound is hydrolyzed.

27. The method as claimed in claim 25, wherein
the further amino acid is added to the new precursor molecule,
the further amino acid has an amino function,
the amino function is protected with a protecting group, and
the method further comprises eliminating the protecting group from the amino function.

28. The method as claimed in claim 27, wherein
activating the carboxyl function of the radioactively labeled compound comprises converting the isocyanocarboxylic acid into a reactive substance which is selected from the group consisting of active ester, anhydride, pentafluorophenyl ester, thioester, imidazolide, acyl halide and dimethylaminopyridine, and
activating the carboxyl function of the further amino acid comprises converting the further amino acid into a reactive substance which is selected from the group consisting of active ester, anhydride, pentafluorophenyl ester, thioester, imidazolide, acyl halide and dimethylaminopyridine.

29. The method as claimed in claim 22, wherein the isocyanocarboxylic acid is an alpha-isocyanocarboxylic acid.

30. The method as claimed in claim 22, wherein activating the carboxyl function of the radioactively labeled compound comprises:
converting the isocyanocarboxylic acid into a reactive substance which is selected from the group consisting of active ester, anhydride, pentafluorophenyl ester, thioester, imidazolide, acyl halide and dimethylaminopyridine.

31. The method as claimed in claim 30, wherein
the isocyanocarboxylic acid is converted to the active ester, and
the isocyanocarboxylic acid is reacted with a coupling reagent selected from the group consisting of a guanidinium reagent, a uronium reagent, a benzotriazole reagent, an immonium reagent, a carbodiimide reagent, an imidazolium reagent, an organophosphorous reagent, an acidic halogenating reagent, a phosphonium reagent, a morpholine reagent, a chloroformate reagent and a combination thereof, to produce the active ester.

32. The method as claimed in claim 30, wherein
the isocyanocarboxylic acid is converted to the active ester, and
the isocyanocarboxylic acid is reacted with a coupling reagent selected from the group consisting of 2-(H-benzotriazol-1-yl)-1,1,3,3-tetramethyluronium hexafluorophosphate (HBTU), O-(benzotriazol-1-yl)-N,N,N′,N′-tetramethyluronium tetrafluoroborate (TBTU), 2-(1H-7-azabenzotriazol-1-yl)-1,1,3,3-tetramethyluronium hexafluorophosphate (HATU), 1-hydroxybenzotriazole reagent (HOBt), N,N′-dicyclohexyl carbodiimide (DCC), diisopropyl carbodiimide (DIPCDI), benzotriazol-1-yl-oxy-tris(dimethylamino)phosphonium hexafluorophosphate (BOP), benzotriazol-1-yl-oxytripyrrolidinophosphonium hexafluorophosphate (PyBOP), N-methylmorpholine (NMM), and a combination thereof, to produce the active ester.

33. The method as claimed in claim 22, wherein
the precursor molecule and/or the isocyanocarboxylic acid has a side chain function selected from the group consisting of a hydroxyl function, a carboxyl function and an amino function, and
the side chain function is blocked by a protecting group.

34. The method as claimed in claim 22, wherein
the precursor molecule has a main chain with a carboxyl function and/or an amino function, and
the carboxyl function and/or the amino function of the main chain of the precursor molecule is/are blocked by a protecting group.

35. The method as claimed in claim 33, wherein the protecting group is selected from the group consisting of a base-stable protecting group, a t-butyloxycarbonyl protecting group and a 9-fluorenylmethoxycarbonyl protecting group.

36. The method as claimed in claim 22, wherein
the precursor molecule has a main chain with an amino function, the amino function being blocked with a protecting group, and
the following steps are repeated until a desired size of the peptide is achieved:
eliminating the protecting group from the amino function of the main chain of the precursor molecule;
adding the further amino acid having a main chain with a protected amino group, to the precursor molecule;
activating a carboxyl function of the main chain of the further amino acid; and
after activating the carboxyl function, linking the precursor molecule to the further amino acid via an amide bond.

37. The method as claimed in claim 35, wherein
the protecting group is a 9-fluorenylmethoxycarbonyl protecting group, and
the 9-fluorenylmethoxycarbonyl protecting group is eliminated with ammonia, a primary amine or a secondary amine.

38. The method as claimed in claim 35, wherein
the protecting group is a 9-fluorenylmethoxycarbonyl protecting group, and
the 9-fluorenylmethoxycarbonyl protecting group is eliminated with 4-aminomethylpiperidine, piperidine or tris(2-aminoethyl)amine.

39. The method as claimed in claim 35, wherein
the protecting group is a t-butyloxycarbonyl protecting group, and
the t-butyloxycarbonyl protecting group is eliminated by protons.

40. The method as claimed in claim 22, additionally comprising the step:
hydrolyzing the activated radioactively labeled compound linked to the precursor molecule, to form an amino group from an isocyano group of the isocyanocarboxylic acid.

41. The method as claimed in claim 22, wherein
a protecting group is attached to the radioactively labeled peptide, and
the method further comprises eliminating the protecting group from the radioactively labeled peptide.

42. The method as claimed in claim 22, wherein the precursor molecule and the radioactively labeled peptide are coupled to a solid phase support.

43. The method as claimed in claim 42, wherein the solid phase support is selected from the group consisting of a polystyrene resin, a 2′,4′-dimethoxyphenylhydroxymethyl-phenoxy resin, a p-methylbenzhydrylamine resin, a phenalacetamidomethyl resin and an oxime resin.

44. The method as claimed in claim 42, further comprising decoupling the radioactively labeled peptide from the solid phase support.

45. The method as claimed in claim 44, wherein
the method further comprises performing hydrolysis on the activated radioactively labeled compound linked to the precursor molecule, to form an amino group from an isocyano group of the isocyanocarboxylic acid, and
hydrolysis takes place simultaneously with decoupling the the radioactively labeled peptide from the solid phase support.

46. The method as claimed in claim 44, wherein
a protecting group is attached to the radioactively labeled peptide, and
the method further comprises:
eliminating the protecting group from the radioactively labeled peptide; and
performing hydrolysis on the activated radioactively labeled compound linked to the precursor molecule, to form an amino group from an isocyano group of the isocyanocarboxylic acid, and
the hydrolysis takes place simultaneously with decoupling of the radioactively labeled peptide from the solid phase support and eliminating the protecting group.

47. The use of a radioactively labeled isocyanocarboxylic acid for producing a radioactively labeled peptide.
